(19) United States
(12) Patent Application Publication
(10) Pub. No.: US 2021/0113147 A1
(43) Pub. Date: Apr. 22, 2021

(54) JAUNDICE DIAGNOSIS PATCH, PATCH AUTHENTICATION SYSTEM, AND JAUNDICE DIAGNOSIS METHOD USING THE SAME

(71) Applicant: AJOU UNIVERSITY INDUSTRY-ACADEMIC COOPERATION FOUNDATION, Suwon-si (KR)

(72) Inventors: Myung Hoon SUNWOO, Seoul (KR); Gi Jo AN, Suwon-si (KR)

(73) Assignee: AJOU UNIVERSITY INDUSTRY-ACADEMIC COOPERATION FOUNDATION, Suwon-si (KR)

(21) Appl. No.: 17/071,477

(22) Filed: Oct. 15, 2020

(30) Foreign Application Priority Data
Oct. 17, 2019 (KR) 10-2019-0129409

(51) Int. Cl. A61B 5/00; A61B 5/1171; G06F 21/32

(52) U.S. Cl. CPC A61B 5/441; A61B 5/683; G06F 21/32; A61B 5/1176; A61B 5/6821; A61B 5/48

(57) ABSTRACT

As a method for diagnosing jaundice of a user based on an image by a jaundice diagnosis device, a jaundice diagnosis image is obtained including a jaundice diagnosis reference device divided into a first area and a second area, and a specific body part of a user located in the first area. The identification information of the jaundice diagnosis reference device inserted in the second area is extracted, and validity authentication of the identification information is requested through an interlocked authentication system. When the validity authentication success message is received from the authentication system, the user is diagnosed with jaundice based on the image of a specific body part located in the first area and the jaundice diagnosis comparison image included in the second area.

### CROSS-REFERENCE TO RELATED APPLICATION

This application is based on and claims priority under 35 U.S.C. § 119 to Korean Patent Application No. 10-2019-0129409, filed on Oct. 17, 2019, in the Korean Intellectual Property Office, the disclosure of which is incorporated by reference herein in its entirety.

### BACKGROUND

### 1. Field

The present disclosure relates to a jaundice diagnosis patch, a patch authentication system, and a jaundice diagnosis method using the same.

### 2. Description of Related Art

Jaundice refers to yellow pigmentation in the whites of the eyes (sclera), skin, and mucous membranes when yellow bile pigment (bilirubin), which is produced in the process of decomposing special proteins containing iron such as hemoglobin in the body, accumulates more than necessary in the body. Existing diagnostic methods for diagnosing jaundice based on scientific view or serological tests have several problems.

When jaundice is diagnosed through examination, which is a scientific test for diagnosing jaundice with the naked eye, there is a problem that it is subjective and cannot provide an objective diagnosis. Therefore, in order to confirm the diagnosis of jaundice, the patient's blood must be collected and the bilirubin level in the blood must be measured.

In this case, there is a disadvantage in that the diagnosis is possible only when a patient with suspected jaundice visits a medical institution equipped with a device capable of measuring the blood bilirubin level, and there is a disadvantage in that it takes a long time to obtain the test result after blood collection.

In order to alleviate such hassle, patches are being distributed so that suspected jaundice patients can self-diagnose jaundice. The image-based jaundice diagnosis system aims to minimize the change in color according to the surrounding light source through the patch.

However, there is a disadvantage in that the current jaundice diagnosis system using a patch does not recognize the patch well. The problem that the jaundice diagnosis system does not recognize the patch causes a major problem with the white balance function to restore color distortion so that the jaundice diagnosis system does not operate properly.

In addition, there is a disadvantage in that it is difficult to give a feeling of satisfaction in terms of user experience, since the user has to find the sclera of the eye and input it into the jaundice diagnosis system.

The current jaundice diagnosis system identifies patients through a user's personal device, and the patch can be used continuously once purchased. However, in the application installed on the user's personal device, since the patient's personal data and history are managed according to the logged-in ID, if a user logs in with someone else's ID and takes a shoot, personal management of data and history is not possible.

### SUMMARY

Accordingly, the present disclosure provides a jaundice diagnosis patch, a patch authentication system, and a jaundice diagnosis method using the same, which can limit a system user to a user by using an improved color correction patch and biometric information for identifying an individual.

Additional aspects will be set forth in part in the description which follows and, in part, will be apparent from the description, or may be learned by practice of the presented embodiments of the disclosure.

As a jaundice diagnosis device for diagnosing jaundice of a user based on an image, which is one feature of the present disclosure for achieving the technical problem of the present disclosure, includes

a photographing unit configured to photograph a jaundice diagnosis reference device divided into a first area and a second area, and a specific body part of the user located in the first area as a jaundice diagnosis image;

an interface configured to transmit identification information extracted from the jaundice diagnosis image to a patch authentication system in order for validity authentication of the jaundice diagnosis reference device; and

a processor configured to extract the identification information by recognizing the second region from the jaundice diagnosis image, extract an image of the specific body part from the first area when the patch authentication system receives a validity authentication signal of the jaundice diagnosis reference device, and diagnose jaundice of the user based on the extracted specific body part image and the second region image.

The jaundice diagnosis device may further include an input means configured to collect the biometric information of the user, wherein the processor may transmit the collected biometric information to the patch authentication system through the interface to request authentication for the user.

The processor may extract the identification information from the third pattern area into which the identification information is inserted in the second area in which the first pattern area of a specific color, the second pattern area of white, and the third pattern area of the specific color are intersected.

The processor may generate a correction image by correcting a distorted color of the jaundice diagnosis image, and extract an image of the specific body part from the generated correction image.

As a method for diagnosing jaundice of a user based on an image by a jaundice diagnosis device, which is another feature of the present disclosure for achieving the technical problem of the present disclosure, includes

obtaining a jaundice diagnosis image photographed including a jaundice diagnosis reference device divided into a first area and a second area, and a specific body part of the user located in the first area;

extracting identification information of the jaundice diagnosis reference device inserted in the second area, and requesting validity authentication of the identification information through an interlocked authentication system; and

when receiving a validity authentication success message from the authentication system, diagnosing whether the user has jaundice based on an image of the specific body part located in the first area and a jaundice diagnosis comparison image included in the second area.

In the second area, a first pattern area of a specific color, a second pattern area of white, and a third pattern area of the specific color may be arranged to intersect each other, wherein the requesting of the validity authentication may include extracting the identification information from the third pattern area into which the identification information is inserted, and transmitting information on a photographing date at which the jaundice diagnosis image is photographed and the identification information to the authentication system.

The diagnosing of whether the user has jaundice may include generating a corrected image by correcting a distorted color of the jaundice diagnosis image, and extracting an image of the specific body part from the generated correction image, and comparing the extracted image with an image of the second pattern area to diagnose jaundice.

The method may further include, before the obtaining of the jaundice diagnosis image, receiving an application driving input signal for diagnosing jaundice of the user, collecting biometric information of the user who executes the application, and transmitting the biometric information to the authentication system to request user authentication for the user.

A jaundice diagnosis reference device for diagnosing whether a user has jaundice based on an image, which is another feature of the present disclosure for achieving the technical problem of the present disclosure, includes

a body part exposure part in which a region of interest drilled in a predetermined size at a first position is formed, and a specific body part of the user located in the region of interest is exposed, and a patch confirmation part formed at a second position spaced apart from the first position by a predetermined interval, and including a pattern formed.

In the patch confirmation part, a first pattern area of a specific color, a second pattern area of white, and a third pattern area of the specific color may be arranged to intersect each other, wherein identification information of the jaundice diagnosis reference device may be inserted in the third pattern area.

The identification information may be inserted in the third pattern area in the form of Quick Response (QR) code or barcode.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is an exemplary diagram of an environment in which jaundice self-diagnosis is possible according to an embodiment of the present disclosure;

FIG. 2 is an exemplary diagram of a jaundice diagnostic patch according to an embodiment of the present disclosure;

FIG. 3 is a structural diagram of a terminal according to an embodiment of the present disclosure;

FIG. 4 is a structural diagram of a patch authentication system according to an embodiment of the present disclosure;

FIG. 5 is a flowchart of a method for self-diagnosing jaundice according to an embodiment of the present disclosure; and

FIG. 6A, FIG. 6B, FIG. 6C, FIG. 6D, FIG. 6E, FIG. 6F, FIG. 6G and FIG. 6H are an exemplary diagram for a method for self-diagnosing jaundice according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments of the present disclosure will be described in detail with reference to the accompanying drawings so that those of ordinary skill in the art may easily implement the embodiments. However, the present disclosure may be implemented in various different forms and is not limited to the embodiments described herein. In the drawings, parts irrelevant to the description are omitted in order to clearly describe the present disclosure, and like reference numerals refer to like elements throughout the specification.

Throughout the specification, when a certain part “includes” a certain component, it means that other components may be further included rather than excluding other components unless otherwise stated.

In the present specification, a terminal may refer to various types of terminals such as mobile, smart phone, and tablet.

Hereinafter, a jaundice diagnosis patch, a patch authentication system, and a jaundice diagnosis method using the same according to an embodiment of the present disclosure will be described with reference to the drawings.

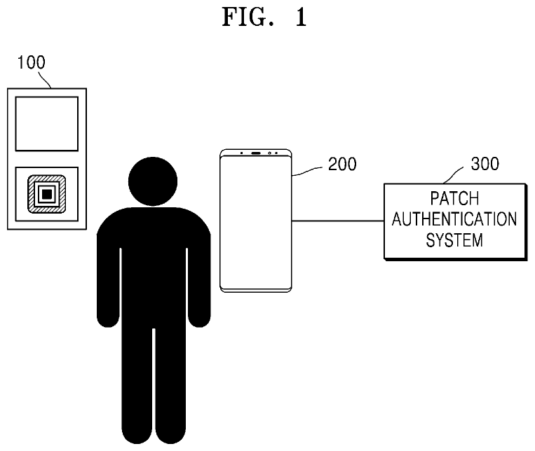

FIG. 1 is an exemplary diagram of an environment in which jaundice self-diagnosis is possible according to an embodiment of the present disclosure.

As shown in FIG. 1, a user may possess a jaundice diagnosis patch 100 and a terminal 200 (or, also referred to as a jaundice diagnosis system), and may self-diagnose whether jaundice occurs using the jaundice diagnosis patch 100 and the terminal 200. In addition, the terminal 200 interoperates with a patch authentication system 300.

The terminal 200 is installed with an application for diagnosing jaundice. When the user places the jaundice diagnosis patch 100 close to the eye and executes the application installed in the terminal 200, the application activates the camera inserted in the terminal 200 to photograph the user's face.

The application of the terminal 200 checks whether there is the jaundice diagnosis patch 100 from an image of a user's face. The application checks whether there is the jaundice diagnosis patch 100 based on the pattern formed on the jaundice diagnosis patch 100. In addition, if the image of the jaundice diagnosis patch 100 is displayed in the image of the user's face, the terminal 200 recognizes the user's eyeball and diagnoses whether jaundice occurs.

The patch authentication system 300 authenticates the user who runs the application and checks the validity of the jaundice diagnosis patch 100 in conjunction with the jaundice diagnosis application installed in the terminal 200.

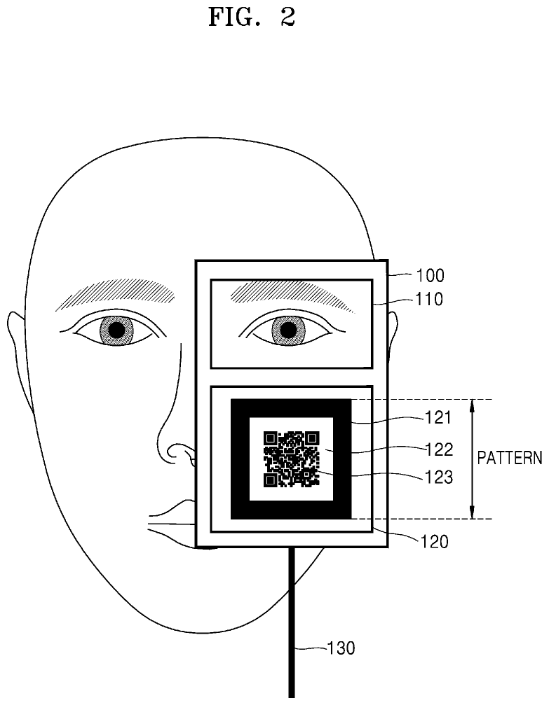

Here, the jaundice diagnosis patch 100 will be described with reference to FIG. 2. In an embodiment of the present disclosure, the jaundice diagnostic patch 100 is used, but may also be referred to as a jaundice diagnostic reference device.

FIG. 2 is an exemplary diagram of a jaundice diagnostic patch according to an embodiment of the present disclosure.

As shown in FIG. 2, the jaundice diagnostic patch 100 is divided into a user's eye position region 110 formed at the first position, and a patch confirmation area 120 at a second position that is the lower end of the first position. When the jaundice diagnosis patch 100 is referred to as a jaundice diagnosis reference device, the user's eye position region 110 may be referred to as an eyeball position part and the patch confirmation region 120 may be referred to as a patch confirmation part.

The user's eye position region 110 is formed on the upper end of the jaundice diagnosis patch 100, and is punctured in a predetermined size so that the user's eye can be checked when the user places the jaundice diagnosis patch 100 close to the face. Here, in the embodiment of the present disclosure, the punctured portion is referred to as a “region of interest”.

When the user's specific body part (eyeball) is located in any region in the region of interest, the application can recognize the pupil from the user's eye and diagnose whether jaundice occurs based on the color of the sclera.

In the patch confirmation region 120, the pattern is formed so that the jaundice diagnosis application can confirm that the patch located on the user's face in the image is the jaundice diagnosis patch 100. The pattern is divided into a first patch region 121, a second patch region 122, and a third patch region 123.

In the embodiment of the present disclosure, in relation to the pattern, a second patch region 122, which is an intermediate white region, is positioned between the first patch region 121, which is an outer black region, and the third patch region 123, which is an inner black region, as an example. The thickness of each of the patch regions 121 to 123 is not limited to any one value.

In addition, a patch ID is inserted into the third patch region 123 for validity verification of the jaundice diagnosis patch 100. In the embodiment of the present disclosure, the patch ID is inserted into the third patch region 123 in the form of a quick response (QR) code or barcode, but is not limited thereto.

A handle 130 is provided at the lower end of the jaundice diagnosis patch 100 so that the user can easily place the jaundice diagnosis patch 100 on the face. However, the present invention is not necessarily limited as described above, and may be implemented in the form of an eye patch that the user can wear on the face.

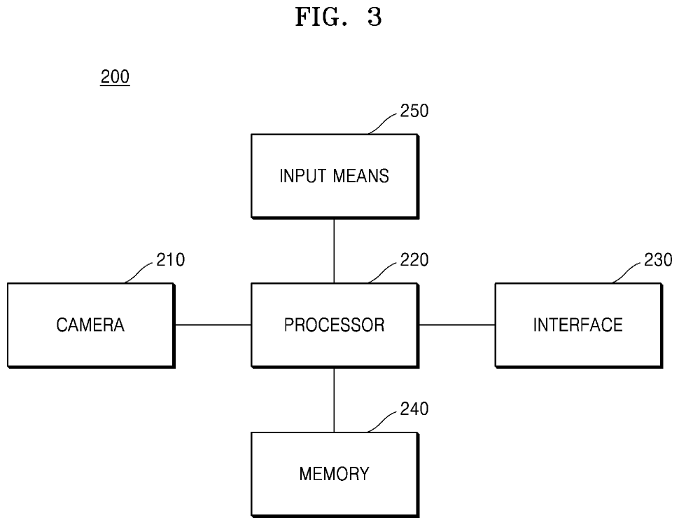

Furthermore, the structure of the terminal 200 that recognizes the jaundice diagnosis patch 100 and diagnoses whether the user has jaundice will be described with reference to FIG. 3. In the embodiment of the present disclosure, for convenience of description, the terminal 200 on which the jaundice diagnosis application is installed is described as diagnosing whether the user has jaundice, but the jaundice diagnosis application performs the jaundice diagnosis.

FIG. 3 is a structural diagram of a terminal according to an embodiment of the present disclosure.

As shown in FIG. 3, a terminal 200 includes a camera 210, a processor 220, an interface 230, a memory 240, and an input means 250.

When the jaundice diagnosis application is executed, the camera 210 becomes active under the control of the processor 220 to capture the jaundice diagnosis patch 100 and the user's face. The image of the user's face in which the jaundice diagnosis patch 100 photographed by the camera 210 is overlapped is referred to as a “patch image”.

In addition, when using iris information for user authentication, the camera 210 may capture a user iris image.

The processor 220 receives the user's personal identification information (hereinafter referred to as “input user information”) inputted through the input means 250. Here, the input user information means iris information or fingerprint information of the user.

The processor 220 compares the user's personal identification information (hereinafter referred to as “stored user information”) stored in the memory 240 with the input user information received through the input means 250. In addition, the user who executes the jaundice diagnosis application installed in the terminal 200 is authenticated. Here, the processor 220 may transmit the input user information received for user authentication to the patch authentication system 300 to perform user authentication in the patch authentication system 300.

The processor 220 generates a control signal to drive the camera 210 when the user who executes the jaundice diagnosis application succeeds in authentication or receives a user authentication success signal from the patch authentication system 300. The generated control signal is transmitted to the camera 210.

The processor 220 extracts a patch confirmation region from the patch image captured by the camera 210. The processor 220 checks the pattern in the extracted patch confirmation region, and checks whether the patch overlapped on the patch image is the jaundice diagnosis patch 100.

When the processor 220 checks the jaundice diagnosis patch 100 from the patch image, the processor 220 extracts the patch ID inserted into the third patch region 123. The processor 220 transmits the extracted patch ID to the patch authentication system 300 through the interface 230.

When the processor 220 receives the validity confirmation information of the jaundice diagnosis patch 100 from the patch authentication system 300, the processor 220 extracts the sclera image of the user's eye photographed in the user's eye position region 110. The processor 220 performs white balancing on the image of the user's eye, and extracts the brightest area into the sclera. Here, since the method of extracting the sclera image from the user's eye by the processor 220 is performed in various ways, the embodiment of the present disclosure is not limited to any one method.

The processor 220 compares the extracted scleral image and the second patch region 122 to analyze whether the user has jaundice. Since the method of analyzing the presence of jaundice by the processor 220 using the scleral image and the second patch region 122 can be performed in various ways, the embodiment of the present disclosure is not limited to any one method.

The interface 230 interworks with the patch authentication system 300 to transmit a patch ID, and receives validity check information transmitted from the patch authentication system 300.

The memory 240 stores an application execution file for driving a jaundice diagnosis application in the terminal 200. In addition, the memory 240 stores jaundice presence information analyzed by the processor 220. In addition, the memory 240 stores various programs required to drive the terminal 200.

The input means 250 collects authentication information for user authentication, such as a user's fingerprint. Since the method of collecting the fingerprint by the input means 250 is various, the embodiment of the present disclosure is not limited to any one method.

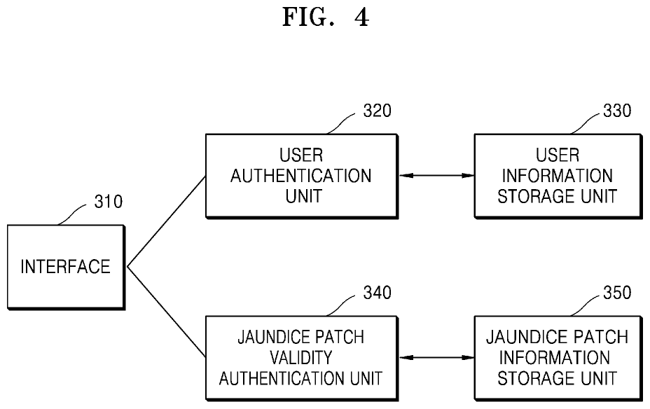

Next, a patch authentication system for authenticating the validity of a jaundice patch or authenticating a user in connection with the terminal 200 will be described with reference to FIG. 4.

FIG. 4 is a structural diagram of a patch authentication system according to an embodiment of the present disclosure.

As shown in FIG. 4, a patch authentication system 300 includes an interface 310, a user authentication unit 320, a user information storage unit 330, a jaundice patch validity authentication unit 340, and a jaundice patch information storage unit 350.

The interface 310 receives user information transmitted from the terminal 200.

When the user's personal identification information is received from the terminal 200 through the interface 310, the user authentication unit 320 compares the personal identification information stored in the user information storage unit 330 with the received user's personal identification information. Based on the comparison result, the user authentication unit 320 checks whether the user executing the jaundice diagnosis application is an approved user.

In the embodiment of the present disclosure, it is described as an example that when the jaundice diagnosis application is executed, by receiving biometric information such as a user's fingerprint or iris as input and comparing the received biometric information with biometric information previously stored in the user information storage unit 330, whether the user is an authorized user is checked. However, the present invention is not limited thereto.

When the user authentication is successful, the user authentication unit 320 generates an authentication success signal and transmits the generated authentication success signal to the terminal 200 through the interface 310. However, if the user authentication fails, the user authentication unit 320 generates an unavailable message indicating that the user cannot use the jaundice diagnosis application and provides the generated message to the terminal 200, or generates an authentication failure signal and transmits the generated signal to the terminal 200.

In addition, the interface 310 receives the jaundice patch identification information transmitted from the terminal 200. The jaundice patch validity authentication unit 340 checks valid information corresponding to the jaundice patch identification information registered in the jaundice patch information storage unit 350.

Here, when the user first attempts to diagnose jaundice using the jaundice diagnosis patch 100, the terminal 200 transmits the jaundice patch identification information of the jaundice diagnosis patch 100 and the date information on which the jaundice patch identification information is collected to the patch authentication system 300. Since all the jaundice diagnosis patches 100 have a predetermined period of use, it is necessary to collect date information at the time when the jaundice diagnosis patch 100 is first used.

If the jaundice patch validity authentication unit 340 confirms that the valid period of the jaundice diagnosis patch 100 remains, it generates a validity confirmation success signal and transmits the generated signal to the terminal 200. Furthermore, if the jaundice patch validity authentication unit 340 confirms that the valid period of the jaundice diagnostic patch 100 expires, it generates an expiration signal for inducing the purchase of a new jaundice diagnosis patch 100 together with a validity check failure signal and transmits the generated signals to the terminal 200.

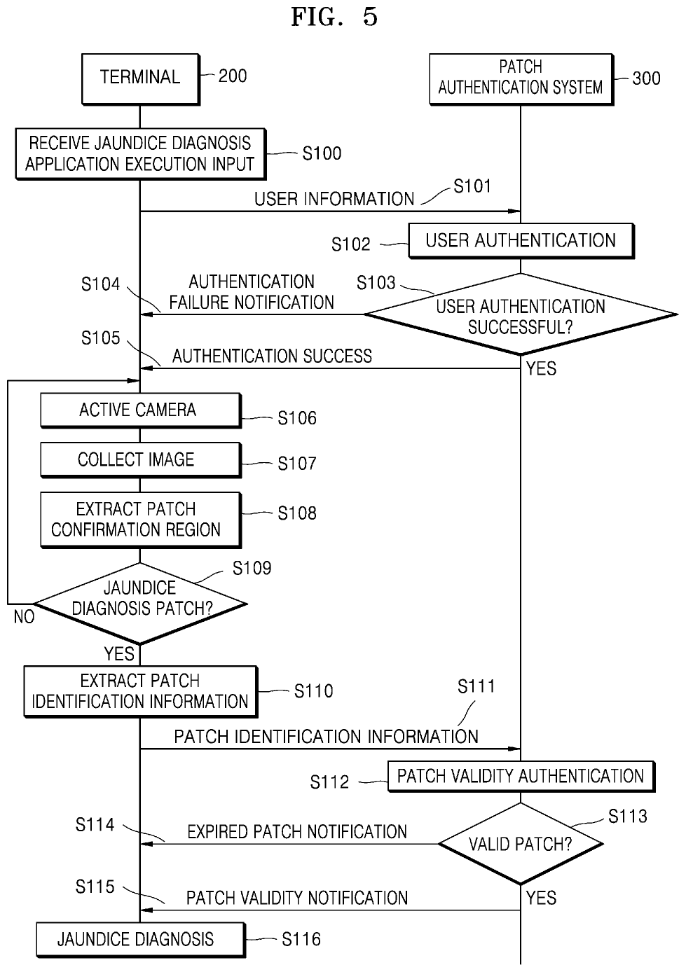

The above-described method of checking the patch validity of the jaundice diagnostic patch 100 and diagnosing whether jaundice occurs by using the jaundice diagnostic patch 100 will be described with reference to FIG. 5.

FIG. 5 is a flowchart of a method for self-diagnosing jaundice according to an embodiment of the present disclosure.

As shown in FIG. 5, when the terminal 200 receives an input signal requesting execution of a pre-installed jaundice diagnosis application (S100), the terminal 200 collects information on a user who executes the application. When the jaundice diagnosis application is executed, the terminal 200 collects the user's biometric information and transmits the collected user information to the patch authentication system 300 (S101).

In the embodiment of the present disclosure, a fingerprint is collected as user information as an example, but iris information or face recognition information of a user may be used. The method of collecting the user's biometric information by the terminal 200 is already known, and the embodiment of the present disclosure is not limited to any one method.

The patch authentication system 300 authenticates the user by comparing the input user information received in operation S101 with the stored user information (S102). The patch authentication system 300 compares the received input user information with pre-stored user information, and checks whether user authentication is successful according to the comparison result (S103).

If the user authentication fails, the patch authentication system 300 transmits a message indicating that the user authentication fails to the terminal 200 (S104). The transmitted message is displayed to the user through the terminal 200.

However, if the user authentication is successful as a result of checking in operation S103, the patch authentication system 300 provides an authentication success signal to the terminal 200 (S105). When the terminal 200 receives an authentication success signal from the patch authentication system 300, the terminal 200 activates the camera (S106). Since the method of activating the camera by the terminal 200 receiving the authentication success signal may be implemented in various ways, the embodiment of the present disclosure is not limited to any one method.

The terminal 200 collects the user face image from the activated camera 210 (S107). Here, the user face image refers to a photographed image in which the jaundice diagnosis patch 100 possessed by the user is placed on a certain part of the face, and is an image photographed in a form in which either one of the user's eyes is located in the user's eye position region 110 of the jaundice diagnosis patch 100.

The terminal 200 extracts a patch confirmation region from the image at a preset position among the user face images collected in operation S107 in order to check whether the pattern of the jaundice diagnosis patch 100 is inserted in the user face image (S108). The patch confirmation region of the jaundice diagnosis patch 100 will be described with an example in which a white second patch region 122 is disposed between the black first patch region 121 and the third patch region 123.

Here, the first patch region 121 and the third patch region 123 may be used to obtain an image in focus, and the second patch region 122 may be used as a reference for correcting the color of the jaundice diagnosis image. In the embodiment of the present disclosure, as shown in FIG. 2, in order to facilitate identification of the jaundice diagnostic patch 100 and other body parts (e.g., the eyeball) of the user, the first patch region 121 and the third patch region 123 are illustrated as an example having color regions intersected with the second patch region 122 interposed therebetween, but the color gamut of the jaundice diagnosis patch 100 may be configured in various forms.

That is, the terminal 100 analyzes an image captured by the camera 210 in real time before image capturing, and uses an edge detection algorithm to detect the edges of the first and third patch regions 121 and 123. At this time, various algorithms such as canny edge detector algorithm, Roberts edge detection algorithm, Sobel edge detection algorithm, and Prewitt edge detection algorithm can be used for the edge detection algorithm, and the embodiment of the present disclosure is not limited to any one algorithm.

When the edges of the first patch region 121 and the third patch region 123 are detected, the terminal 100 determines that the image is in focus, and confirms that the image of the jaundice diagnosis patch 100 is included in the image captured by the camera 210.

In this case, the terminal 100 may provide a capturing guideline through voice, so that a focused image can be captured. For example, if the user is too close, the terminal 100 may output a message to step back a little, and if the user's eyeball 400 is small and difficult to identify, the terminal 100 may output a message to open the eyes wide.

In addition, when a portion of the first patch region 121 is detected as being cut off, the terminal 100 may output a message to move in a direction in which both the edges of the first patch region 121 and the third patch region 123 can be detected.

Moreover, if it is determined that there is no jaundice diagnosis patch 100 in the user's face image based on the patch confirmation region extracted in operation S108, the terminal 200 repeats the procedure after operation S106.

Furthermore, if it is determined that the jaundice diagnosis patch 100 is located in the user's face image in operation S108, the terminal 200 extracts the patch identification information inserted into the third patch region 123 in the patch confirmation regions (S110). In the embodiment of the present disclosure, patch identification information is inserted into the third patch region 123 in various forms, such as a QR code and a barcode, and the method of inserting the patch identification information is not limited to any one method.

The terminal 100 transmits the patch identification information extracted in operation S110 to the patch authentication system 300 together with the date information on which the user's face image is collected (S111). The patch authentication system 300 authenticates the validity of the jaundice diagnosis patch 100 based on the patch identification information and date information received in operation S111 (S112).

For example, it is assumed that the valid period of 1 year is given to all the jaundice diagnostic patches 100 after use of the patch. In addition, date information on when the user starts self-diagnosis of jaundice using the first jaundice diagnosis patch 100 is transmitted to and stored in the patch authentication system 300.

The patch authentication system 300 stores a one-to-one mapping of a plurality of patch identification information and the first use date information. And, upon receiving the patch identification information from the terminal 100, it is checked whether the date information corresponding to the patch identification information is stored.

In order for patch validity authentication in operation S112, it is checked whether there is the patch identification information received in operation S111 among the patch identification information of the previously stored patches. If the patch identification information does not exist, the patch identification information and date information received in operation S111 are mapped one-to-one and stored. And, it is notified to the terminal 100 that the jaundice diagnosis patch 100 is a valid patch.

However, if information corresponding to the received patch identification information is stored, first execution date information is checked based on the patch identification information. And, it is checked whether the date received in operation S111 is within the valid period from the checked date (S113).

If the valid period elapses, the patch authentication system 300 notifies the terminal 100 of the expiration of the valid period of the jaundice diagnosis patch 100 and induces the purchase of a new jaundice diagnosis patch 100 (S114). In order to induce purchase of the new jaundice diagnosis patch 100, URL information of a sales site that sells the jaundice diagnosis patch 100 may be provided.

However, if the valid period of the jaundice diagnosis patch 100 checked in operation S113 is before expiration, a patch validity notification message is transmitted to the terminal 100 (S115). When receiving the patch validity notification message, the terminal 100 diagnoses whether the user has jaundice from the image collected in operation S107 (S116).

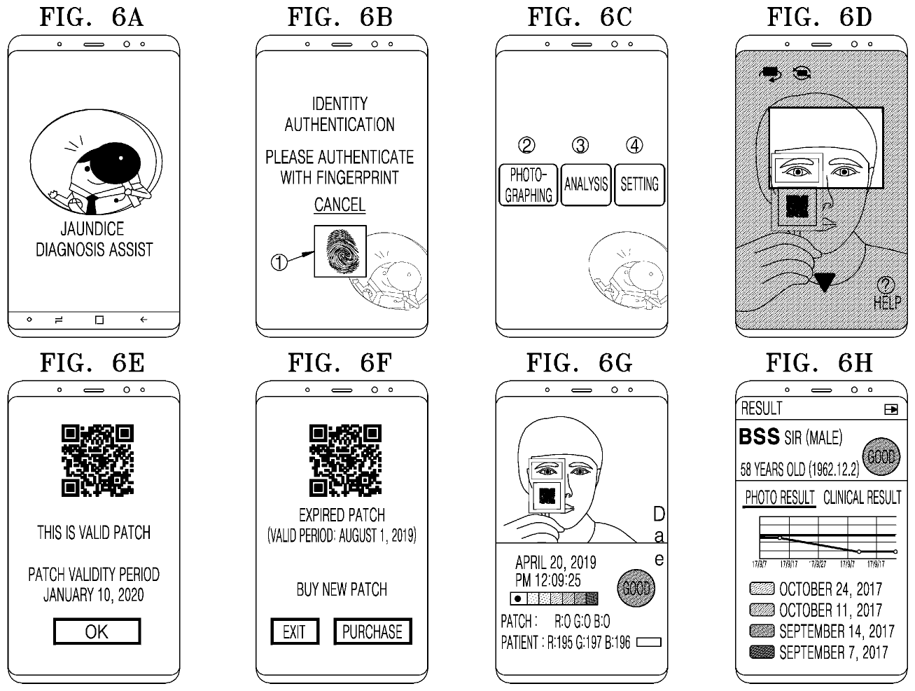

An embodiment of self-diagnosing jaundice through the method described above will be described with reference to FIG. 6A, FIG. 6B, FIG. 6C, FIG. 6D, FIG. 6E, FIG. 6F, FIG. 6G and FIG. 6H

FIG. 6A, FIG. 6B, FIG. 6C, FIG. 6D, FIG. 6E, FIG. 6F, FIG. 6G and FIG. 6H are an exemplary diagram for a method for self-diagnosing jaundice according to an embodiment of the present disclosure.

As shown in FIG. 6A, when the user executes the jaundice diagnosis application installed in the terminal 100, the biometric information collection screen for user authentication is displayed in order to authenticate user information as shown in FIG. 6B. In the embodiment of the present disclosure, authentication of a user through a fingerprint among various biometric information will be described as an example.

For this, when the user first installs the jaundice diagnosis application on the terminal 100, the user's biometric information (e.g., fingerprint, face, iris, etc.) is inputted and registered in the patch authentication system 300. When a user inputs a fingerprint through the biometric information recognition window \textcircled{1} shown in FIG. 6B, the fingerprint information inputted by the user is transmitted to the patch authentication system 300.

When the biometric information of the registered user matches the collected biometric information, the patch authentication system 300 transmits an authentication success signal to the terminal 100. Then, the jaundice diagnosis application provides menus to be selected by the user, as shown in FIG. 6C.

In the embodiment of the present disclosure, photographing \textcircled{2}, analysis \textcircled{3}, and setting \textcircled{4} menus are described as examples, but the selection menu is not limited thereto. When the user selects the photographing \textcircled{2} menu, the camera of the terminal 100 is activated so that the user's face image can be photographed as shown in FIG. 6D.

The terminal 100 detects edges of the first patch region 121 and the third patch region 123 from the user's face image photographed first through an edge detection algorithm. And, when the edges of the first patch region 121 and the third patch region 123 are detected, it is confirmed that the image of the jaundice diagnosis patch 300 is included in the user's face image, and patch identification information inserted in the third patch region 123 is collected.

A patch validity authentication message as shown in FIG. 6E or FIG. 6F is received from the patch authentication system 300 that authenticates the patch validity using the collected patch identification information. If the valid period of the jaundice diagnosis patch 100 remains, as shown in FIG. 6E, a message indicating that the patch is valid and a patch valid period are displayed.

However, if the valid period of the jaundice diagnostic patch 100 elapses, together with information notifying that the valid period of the jaundice diagnosis patch 100 expires, information for purchasing a new jaundice diagnosis patch is provided. In the embodiment of the present disclosure, a purchase button is additionally displayed, but a URL of a purchase site where the jaundice diagnosis patch 100 can be purchased, or an automatic link to the purchase site may be used.

On the other hand, if the validity of the jaundice diagnosis patch 100 is verified, as shown in FIG. 6G, the user's jaundice is diagnosed and provided with an image. That is, after white-balancing the user's face image, the terminal 100 recognizes the pupils from the white-balanced corrected image.

The terminal 100 compares the RGB pixel value of the sclera region in the recognized pupils with the RGB pixel value of the second patch region 122 of the jaundice diagnosis patch 100 to diagnose whether the user has jaundice. The terminal 100 can perform various methods of diagnosing whether the user has jaundice from the corrected image.

In addition, the terminal 100 displays the diagnosis result as shown in FIG. 6G. In this case, the terminal 100 may provide information on a date and time of self-diagnosis of jaundice, information on whether jaundice occurs, and the like, together with a corrected image. When it is determined that the user has jaundice, the terminal 100 may transmit information indicating whether the user has jaundice together with user information to a server (not shown) of a linked medical institution.

Moreover, when the analysis \textcircled{3} menu is selected among the menus shown in FIG. 6C, the terminal 100 displays information on the result of jaundice diagnosis that the user self-diagnoses.

In such a way, through the process of checking whether the user information registered at the time of initial user registration and the user information of the actual user currently being used are correct by the patch authentication system 300, the jaundice diagnosis application can be restricted to the registered user by authenticating the user who is using the jaundice authentication application. Based on this, security for personal data and history can be strengthened.

In addition, it is possible to limit the usable period from the date that the user starts to use the jaundice diagnosis patch through means such as a barcode or QR code capable of recognizing the patch ID of the jaundice diagnosis patch. Through this, by inducing the repurchase of the jaundice diagnosis patch when the period of use is ended, or by inducing the purchase of a subscription for each period of use in the patch authentication system 300 or repurchase of the jaundice diagnosis patch, revenue may be generated.

According to the present disclosure, it is possible to improve the accuracy and user experience of the jaundice diagnosis system.

In addition, financial costs such as operation of a jaundice diagnosis system and production of color correction patches can be solved by creating a profit model.

In addition, in the case of patients, their own data and history can be protected and the false diagnosis rate can be further lowered through the enhanced personal identification function.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A jaundice diagnosis device that diagnoses jaundice of a user based on an image, the device comprising:
a photographing unit configured to photograph a jaundice diagnosis reference device divided into a first area and a second area, and a specific body part of the user located in the first area as a jaundice diagnosis image;
an interface configured to transmit identification information extracted from the jaundice diagnosis image to a patch authentication system in order for validity authentication of the jaundice diagnosis reference device; and
a processor configured to extract the identification information by recognizing the second region from the jaundice diagnosis image, extract an image of the specific body part from the first area when the patch authentication system receives a validity authentication signal of the jaundice diagnosis reference device, and diagnose jaundice of the user based on the extracted specific body part image and the second region image.

2. The jaundice diagnosis device of claim 1, further comprising an input means configured to collect the biometric information of the user,
wherein the processor transmits the collected biometric information to the patch authentication system through the interface to request authentication for the user.

3. The jaundice diagnosis device of claim 2, wherein the processor extracts the identification information from the third pattern area into which the identification information is inserted in the second area in which the first pattern area of a specific color, the second pattern area of white, and the third pattern area of the specific color are intersected.

4. The jaundice diagnosis device of claim 3, wherein the processor generates a correction image by correcting a distorted color of the jaundice diagnosis image, and extracts an image of the specific body part from the generated correction image.

5. A method for diagnosing jaundice of a user by a jaundice diagnosis device based on an image, the method comprising:
obtaining a jaundice diagnosis image photographed including a jaundice diagnosis reference device divided into a first area and a second area, and a specific body part of the user located in the first area;
extracting identification information of the jaundice diagnosis reference device inserted in the second area, and requesting validity authentication of the identification information through an interlocked authentication system; and
when receiving a validity authentication success message from the authentication system, diagnosing whether the user has jaundice based on an image of the specific body part located in the first area and a jaundice diagnosis comparison image included in the second area.

6. The method of claim 5, wherein in the second area, a first pattern area of a specific color, a second pattern area of white, and a third pattern area of the specific color are arranged to intersect each other,
wherein the requesting of the validity authentication comprises extracting the identification information from the third pattern area into which the identification information is inserted, and transmitting information on a photographing date at which the jaundice diagnosis image is photographed and the identification information to the authentication system.

7. The method of claim 6, wherein the diagnosing of whether the user has jaundice comprises:
generating a corrected image by correcting a distorted color of the jaundice diagnosis image; and
extracting an image of the specific body part from the generated correction image, and comparing the extracted image with an image of the second pattern area to diagnose jaundice.

8. The method of claim 5, further comprising: before the obtaining of the jaundice diagnosis image,
receiving an application driving input signal for diagnosing jaundice of the user;
collecting biometric information of the user who executes the application; and
transmitting the biometric information to the authentication system to request user authentication for the user.

9. A jaundice diagnosis reference device that diagnoses whether a user has jaundice based on an image, the device comprising:
a body part exposure part in which a region of interest drilled in a predetermined size at a first position is formed, and a specific body part of the user located in the region of interest is exposed; and
a patch confirmation part formed at a second position spaced apart from the first position by a predetermined interval, and including a pattern formed.

10. The jaundice diagnosis reference device of claim 9, wherein in the patch confirmation part, a first pattern area of a specific color, a second pattern area of white, and a third pattern area of the specific color are arranged to intersect each other, wherein identification information of the jaundice diagnosis reference device is inserted in the third pattern area.

11. The jaundice diagnosis reference device of claim 10, wherein the identification information is inserted in the third pattern area in the form of Quick Response (QR) code or barcode.
